(19) United States
(12) Patent Application Publication
(10) Pub. No.: US 2026/0364217 A1
(43) Pub. Date: Nov. 19, 2026

(54) PHENOLIC RESIN MICROSPHERE, POROUS CARBON MICROSPHERE, RADIOACTIVE MICROSPHERE, METHOD FOR PREPARING SAME, AND USE THEREOF

(71) Applicant: Chengdu Shetai Medical Technology Co., Ltd., Chengdu, Sichuan (CN)

(72) Inventors: Weikun TANG, Chengdu (CN); Xiaoyan WU, Chengdu (CN); Shuqun WANG, Chengdu (CN)

(73) Assignee: Chengdu Shetai Medical Technology Co., Ltd., Chengdu, Sichuan (CN)

(21) Appl. No.: 19/138,355

(22) PCT Filed: Nov. 30, 2023
(86) PCT No.: PCT/CN2023/135471

§ 371 Date: Jun. 12, 2025

(30) Foreign Application Priority Data
Dec. 13, 2022 (CN) 202211592390.7
Dec. 13, 2022 (CN) 202211592412.X
Dec. 13, 2022 (CN) 202211601750.5

(51) Int. Cl. A61K 51/12

(52) U.S. Cl. CPC A61K 51/1251

(57) ABSTRACT

Disclosed are a phenolic resin microsphere, a porous carbon microsphere, a radioactive microsphere, a method for preparing same, and use thereof. The phenolic resin microsphere is obtained by mixing and reacting a phenolic compound, water, an aldehyde compound, a basic catalyst, and a dispersion medium. The dispersion medium is selected from alkane, cycloalkane, silicone oil, thermally conductive oil, maize oil, peanut oil, olive oil, and industrial white oil. The dispersion medium is free of micromolecular surfactants. The porous carbon microsphere prepared from the phenolic resin microsphere provided by the present invention can be used for carrying a drug or nuclide, possessing benefits including a high loading rate, minimal loss, and enhanced safety.

The present invention claims the benefits of priorities to Chinese Patent Application No. 2022115923907, filed on Dec. 13, 2022, Chinese Patent Application No. 202211592412X, filed on Dec. 13, 2022, and Chinese Patent Application No. 2022116017505, filed on Dec. 13, 2022. The entire contents of the above-mentioned Chinese patent applications are hereby incorporated by reference into the present invention.

### FIELD OF THE INVENTION

The present invention relates to the medical field, and specifically to phenolic resin microsphere, porous carbon microsphere, radioactive microsphere, and method for preparing the same and use thereof.

### BACKGROUND OF THE INVENTION

Malignant tumors are a class of diseases that pose a serious threat to human health, and a variety of treatments are available currently, including chemotherapy, radiation therapy, interventional therapy, and bioimmunotherapy. As one of these therapies, greater progress has been made in localizing radioactive materials provided to cancer patients. Radioactive materials are incorporated into small particles that can be implanted directly into solid cancerous tumors, and the α or β rays released by the radioactive elements are used to achieve localized cell killing, reduce the impact on normal cells surrounding the tumor cells, and maximize the safety of the treatment while ensuring its effectiveness.

In recent years, carbon material microspheres have shown potential applications in adsorption, catalysis, drug delivery, and energy storage. Carbon materials are generally categorized into three types: solid carbon material microspheres, hollow carbon material microspheres, and porous carbon material microspheres. Wherein, porous carbon material microspheres have excellent properties such as high specific surface area, high chemical stability, high adsorption, etc., and have a wide range of applications in batteries, adsorption, and other fields. Carbon material microspheres as adsorption carriers for loading drugs and even radioactive elements have been studied to some extent, but the disclosed content is relatively small and still needs further research.

Meanwhile, as one of the important precursors for the preparation of porous carbon material microspheres, phenolic resin microspheres are characterized by the stability of raw materials, inexpensive price, and high rate of carbon formation, but the raw materials of phenolic resin microspheres, whether commercially available products or reported in the literature, are basically of nanometer level or particle size higher than 100 μm or more. At present, phenolic resin microspheres are often prepared by mixing linear phenolic resin and curing agent, and then crushed, dispersed, and cured at high temperature to prepare phenolic resin microspheres, which is a cumbersome process, and the diameter of the microspheres is too high, which is often more than 100 μm. The commonly used hydrothermal preparation reaction also requires high temperature and high pressure, the reaction time is up to 12 h or more, it is also difficult to break the limit of solid content (generally at 5% or lower), and thus the cost of industrialized production is too high.

Therefore, there is an urgent need to provide a phenolic resin microsphere and preparation method thereof to serve as precursor for the preparation of a porous carbon microsphere for an adsorption carrier for loading a drug or a radioactive element and realizing effects of high loading rate and low loss rate of the radioactive element.

### SUMMARY OF THE INVENTION

The problem to be solved by the present invention is to overcome the defects of carbon microspheres in the prior art, which are unable to simultaneously realize high loading rate and low loss rate of radioactive elements, and to provide a phenolic resin microsphere, a porous carbon microsphere, a radioactive carbon microsphere, and a preparation method and use thereof. The radioactive carbon microsphere of the present invention has a high loading rate and a low static loss rate and vibration loss rate for radionuclide, which effectively improves the safety of the radioactive carbon microsphere during transportation and storage and enhances the possibility of medical use of the radioactive carbon microsphere. At the same time, the phenolic resin microsphere of the present invention has uniform particle size distribution and high yield; the preparation method for phenolic resin microsphere has advantages of good particle size control, fewer steps, low cost, no need to use curing agent and pore-forming agent, etc., which reduces the difficulty of the process and the risk of the production process; the porous carbon microsphere produced by using the phenolic resin microsphere of the present invention has controllable particle size, which is suitable for the pharmaceutical field (e.g., loading of metal element), and has the significant advantages of high loading rate, low loss rate and high safety.

In order to achieve the above objectives, the present invention provides the following technical solutions.

Technical solution 1 provided by the present invention is: a radioactive carbon microsphere. The radioactive carbon microsphere includes a porous carbon microsphere and a complex containing radionuclide;
wherein, the porous carbon microsphere has a mesopore structure, the mesopore has an average pore size of 2 to 15 nm; the complex containing radionuclide is distributed within the pores of the mesopore; and
the porous carbon microsphere has a specific surface area of 6 to 30 m²/g.

In the present invention, the porous carbon microsphere may have a particle size of 1 to 150 μm, preferably 1 to 100 μm, more preferably 10 to 60 μm (e.g., 20 to 50 μm).

In the present invention, the porous carbon microsphere may have a specific surface area of 10 to 30 m²/g, preferably 12 to 26 m²/g, such as 10.01 m²/g, 14.5 m²/g, 22.3 m²/g, and 25.5 m²/g.

In the present invention, the mesopore may have an average pore size of 5 to 10 nm, preferably 6 to 9 nm, such as 6.44 nm, 6.75 nm, 8.48 nm, or 9.32 nm.

In the present invention, in the complex containing radionuclide, the radionuclide may be selected from one or more of ⁹⁰Y, ³²P, ¹⁹²Ir, ¹⁰³Pd, ⁸⁹Sr, ²²⁶Ra, ¹³¹I, ¹²⁵I, ¹⁸⁸Re, ¹⁸⁶Re, ¹⁵³Sm, ¹⁶⁶Ho, ¹¹¹In, ^{99m}Tc, ¹⁹²Ir, ²²⁶Ra, ¹⁷⁷Lu, ²²⁵Ac, ²¹²Bi, ²¹³Bi and ²²³Ra, preferably ⁹⁰Y.

In the present invention, the amount ratio of the porous carbon microsphere to the radionuclide is preferably 1:(0.033 to 0.233), more preferably 1:(0.067 to 0.233), further preferably 1:(0.1 to 0.233), and still further preferably 1:(0.113 to 0.167). For example, when the porous carbon microsphere has a mass of 0.15 g, the mass of the radionuclide contained in the complex is 5 to 35 mg; or, when the porous carbon microsphere has a mass of 0.15 g, the mass of the radionuclide contained in the complex is 10 to 35 mg; or, when the porous carbon microsphere has a mass of 0.15 g, the mass of the radionuclide contained in the complex is 15 to 35 mg; or, when the porous carbon microsphere has a mass of 0.15 g, the mass of the radionuclide contained in the complex is 17 to 25 mg.

In the present invention, the complex containing radionuclide is preferably obtained by reacting a solution comprising radionuclide and a solution comprising a precipitating agent.

Wherein, when the radionuclide is ⁹⁰Y, the solution comprising radionuclide is preferably a ⁹⁰YCl₃ solution.

In the solution comprising precipitating agent, the precipitating agent is preferably one or more of tartaric acid, EDTA and sodium phosphate; more preferably tartaric acid, or EDTA, or tartaric acid and sodium phosphate. The EDTA is preferably EDTA-2Na.

Technical solution 2 provided by the present invention is: a method for preparing radioactive carbon microsphere. The method for preparing the radioactive carbon microsphere comprises the steps in Mode I or Mode II below:
Mode I: Mixing the porous carbon microsphere and a solution containing radionuclide, then adding a solution containing a precipitating agent to obtain a reaction solution, and reacting, in order to obtain the radioactive carbon microsphere; and
Mode II: Mixing the porous carbon microsphere and a solution containing a precipitating agent, then adding a solution containing radionuclide to obtain a reaction solution, and reacting, in order to obtain the radioactive carbon microsphere.

In the present invention, in Mode I and Mode II, the porous carbon microsphere has a mesopore structure, and the mesopore has an average pore size of 2 to 15 nm; and the specific surface area of the porous carbon microsphere is all 6 to 30 m²/g.

In the present invention, the concentration of the radionuclide in the reaction solution may be 0.02 to 0.15 mol/L, preferably 0.05 to 0.12 mol/L.

In the present invention, preferably, the molar concentration of the precipitating agent in the reaction solution is in excess relative to the molar concentration of the radionuclide in the reaction solution, more preferably by a factor of 1.5 to 4, e.g., the molar concentration of the precipitating agent is 2 times, 2.3 times, 2.5 times, 2.8 times, 3 times, 3.2 times, 3.5 times, or 3.8 times of that of the radionuclide.

In the present invention, the solution comprising precipitating agent may be one or more of a tartaric acid solution, an EDTA solution and a sodium phosphate solution; preferably a tartaric acid solution, or an EDTA solution, or a mixture of a tartaric acid solution and a sodium phosphate solution. The EDTA solution is preferably an EDTA-2Na solution.

In the present invention, after completion of the reaction, the complex containing radionuclide may be distributed in the pores of the mesopore; preferably, the complex containing radionuclide is immobilized in the pores of the mesopore.

In the present invention, the duration of the reaction may be 0.5 to 1.5 h, for example 0.6 h, 0.7 h, 0.8 h, 0.9 h, 1 h, 1.1 h, 1.2 h, 1.3 h or 1.4 h.

In the present invention, the reaction may further comprise the step of cleaning after completion of the reaction. The operation and condition of the cleaning may be conventional in the art, such as repeated rinsing with deionized water.

In the present invention, the method for preparing porous carbon microsphere preferably comprises the step of calcining carbonization of resin microsphere (e.g., phenolic resin microsphere or polystyrene microsphere).

Wherein, the temperature of the calcining carbonization is preferably 400 to 1000 °C and further may be 400 to 800 °C (e.g., 700 to 800 °C). In the process of elevating temperature from room temperature to calcination temperature (e.g., 400 to 800 °C), the rate of elevating temperature may be conventional in the art, such as 3 °C/min to 10 °C/min.

Wherein, the duration of the calcining carbonization may be 1 to 10 h, preferably 3 to 6 h, more preferably 2 to 4 h.

Wherein, the atmosphere for the calcining carbonization is preferably an Ar gas atmosphere.

In the present invention, the method for preparing porous carbon microsphere may further comprise the steps of cleaning, drying, and sieving after the end of the carbonization, according to the convention of the art.

Wherein, the cleaning may be conventional in the art, preferably cleaning with diluted nitric acid (e.g., 0.3 mol/L of diluted nitric acid) and deionized water in sequence.

Wherein, the drying may be conventional in the art, preferably vacuum drying or blast drying oven drying.

Wherein, the sieving may be conventional in the art, preferably by means of a sifter.

In the present invention, the method for preparing porous carbon microsphere preferably comprises the following steps:

Placing the phenolic resin microsphere in a tube furnace and carbonizing under an inert atmosphere; at the end of carbonization, cleaning, drying and sieving to obtain the porous carbon microsphere.

The technical solution 3 provided by the present invention is: a method for preparing a phenolic resin microsphere. The method for preparing the phenolic resin microsphere comprises mixing and reacting a phenolic compound, water, an aldehyde compound, a basic catalyst, and a dispersion medium to obtain the phenolic resin microsphere;

Wherein, the dispersion medium is selected from one or more of alkane, cycloalkane, silicone oil, heat transfer oil, corn oil, peanut oil, olive oil, and industrial white oil; and the dispersion medium is free of small molecular surfactant.

In the present invention, the mixing may be conventional in the art, e.g., mixing the phenolic compound with deionized water before adding a deionized water solution of the basic catalyst, formaldehyde, and a dispersion medium.

In the present invention, the mixing can generally be mixed well under stirring condition.

Wherein, the stirring may be conventional in the art, such as magnetic stirring.

The rate of the stirring is preferably 50 to 400 rpm/min, further may be 50 to 300 rpm/min or 70 to 400 rpm/min, further may be 100 to 300 rpm/min, 150 to 200 rpm/min or 100 to 200 rpm/min.

When the dispersion medium is a hydrocarbon organic solvent (the alkane, cycloalkane), the rate of the stirring is preferably 50 to 300 rpm/min, more preferably 100 to 200 rpm/min. When the dispersion medium is an oil organic solvent (the silicone oil, heat transfer oil, corn oil, peanut oil, olive oil and industrial white oil), the rate of the stirring is preferably 70 to 400 rpm/min, more preferably 100 to 300 rpm/min and further preferably 150 to 200 rpm/min.

The duration of the stirring is preferably 20 to 60 min, more preferably 30 to 40 min.

In the present invention, the temperature of the reaction may be from 50 to 100 °C, preferably from 60 to 85 °C.

The duration of the reaction may be 24 to 72 h, for example 48 h.

In the present invention, the method for preparing the phenolic resin microsphere may further comprise the steps of solid-liquid separation, cleaning and drying after the end of the reaction, according to the convention in the art.

Wherein, the solid-liquid separation may be conventional in the art, preferably filtration or rotary evaporation.

Wherein, the cleaning may be conventional in the art, preferably using anhydrous ethanol and deionized water.

Wherein, the drying may be conventional in the art, preferably vacuum drying or blast drying oven drying.

In the present invention, the alkane may be those known in the art, preferably one or more of n-pentane, n-hexane, n-heptane, and n-octane, more preferably n-heptane.

In the present invention, the cycloalkane may be those known in the art, preferably cyclohexane.

In the present invention, the small molecular surfactant may be those known in the art, generally those having a molecular weight of less than 1,000, such as one or more of span-type surfactant (e.g., Span-80), tween-type surfactant, sulfopropyl betaine-type surfactant (e.g., cetylsulfopropyl betaine), sorbitan monooleate-type surfactant, sodium cocoyl propionate, cetyltrimethylamine bromide (CTAB), dodecyl polyoxyethylene ether-9 and polyethylene glycol octyl phenyl ether.

In the present invention, the basic catalyst may be those known in the art, preferably an inorganic base. The inorganic base may be one or more of aqueous ammonia, metal hydroxide, carbonate, and bicarbonate, preferably one or more of metal hydroxide, carbonate, and bicarbonate.

Wherein, the metal hydroxide may be those known in the art, preferably NaOH, KOH or Ca(OH)₂.

Wherein, the carbonate may be those known in the art, preferably Na₂CO₃, K₂CO₃ or Cs₂CO₃.

Wherein, the bicarbonate may be those known in the art, preferably NaHCO₃ or KHCO₃.

The basic catalyst is preferably one or more of NaOH, KOH, Ca(OH)₂, Na₂CO₃ and K₂CO₃, more preferably Na₂CO₃ or Ca(OH)₂.

In the present invention, the basic catalyst is generally provided in the form of an aqueous solution thereof.

In the present invention, the molar ratio of the phenolic compound to the basic catalyst may be 100:(0.001 to 0.1), preferably 100:(0.005 to 0.05), more preferably 100:0.01.

In the present invention, the phenolic compound may be those known in the art, generally referring to phenolic compound having or not having an electron-donating substituent on the benzene ring, preferably one or more of resorcinol, phenol, cresol, nonylphenol, arylalkylphenol, cashew nut phenol, octylphenol, bisphenol A, and dimethylphenol; more preferably resorcinol or phenol.

In the present invention, the aldehyde compound may be those known in the art, preferably selected from one or more of formaldehyde, acetaldehyde, and furfural, more preferably formaldehyde.

In the present invention, the aldehyde compound is generally provided in the form of an aqueous solution thereof, e.g., 37% aqueous solution of formaldehyde.

In the present invention, the molar ratio of the phenolic compound to aldehyde compound may be 1:(1 to 5), preferably 1:(2 to 4), more preferably 1:3.

In the present invention, the weight-to-volume ratio (w/v) of the phenolic compound to the water may be 1:(3 to 8) g/mL, preferably 1:(4.7 to 7) g/mL, more preferably 1:(5 to 6.25) g/mL.

In the present invention, the volume ratio of the water to the dispersion medium may be 1:(0.5 to 6), further 1:(0.75 to 6) or 1:(0.5 to 4), further 1:(1 to 5), more preferably 1:(2 to 4), further preferably 1:(2.4 to 3).

When the dispersion medium is a hydrocarbon organic solvent (the alkane, cycloalkane), the volume ratio of the water to the dispersion medium may be 1:(0.75 to 6), preferably 1:(1 to 5), more preferably 1:(2 to 4), further preferably 1:(2.4 to 3). When the dispersion medium is an oil organic solvent (the silicone oil, heat transfer oil, corn oil, peanut oil, olive oil and industrial white oil), the volume ratio of the water to the dispersion medium may be 1:(0.5 to 4), preferably 1:(1 to 3), for example 1:1.67 or 1:2.

In the present invention, the water may be conventional deionized water in the art. Unless otherwise specified, the “water” in “the weight-to-volume ratio of the phenolic compound to the water” and “the volume ratio of the water to the dispersion medium” includes the total volume of “water” used in the configuration of an aqueous solution of the phenolic compound and the configuration of an aqueous solution of the basic catalyst (when the basic catalyst is provided in the form of an aqueous solution thereof).

In the present invention, the phenolic resin microsphere is prepared generally without the addition of a curing agent and/or a pore-forming agent. Wherein, the curing agent may be those known in the art, such as hexamethylenetetramine, melamine, etc. The pore-forming agent may be those known in the art, such as toluene, DOP, octadecanol, etc.

In the present invention, the method for preparing the phenolic resin microsphere may comprise the following steps:
(1) Mixing a phenolic compound, water, basic catalyst, aldehyde compound and dispersion medium under stirring condition to obtain a mixture; the dispersion medium is selected from alkane and/or cycloalkane;
Preferably, the molar ratio of the phenolic compound to the basic catalyst is 100:(0.001 to 0.1); the volume ratio of the water to the dispersion medium is 1:(0.75 to 6); and the rate of the stirring is 50 to 300 rpm/min;
(2) Placing the mixture obtained in step (1) in a reactor for reaction;
Preferably, the reaction temperature is 50 to 100 °C; and
(3) After the end of the reaction in step (2), conducting solid-liquid separation, washing, and drying, to obtain the phenolic resin microsphere.

In the present invention, the method for preparing the phenolic resin microsphere may comprise the following steps:
(1) Mixing a phenolic compound, basic catalyst, water, aldehyde compound and a dispersion medium under stirring condition to obtain a mixture; the dispersion agent is selected from one or more of silicone oil, heat transfer oil, corn oil, peanut oil, olive oil and industrial white oil;
Preferably, the rate of the stirring is 70 to 400 rpm/min, the molar ratio of the phenolic compound to the basic catalyst is 100:(0.001 to 0.1); and the volume ratio of the water to the dispersion medium is 1:(0.5 to 4);
(2) Placing the mixture in a reactor for reaction; and
(3) After the end of the reaction in step (2), conducting solid-liquid separation, washing, and drying, to obtain phenolic resin microsphere.

The technical solution 4 provided by the present invention is: a phenolic resin microsphere. The phenolic resin microsphere is produced by the method for preparing a phenolic resin microsphere as previously described.

In the present invention, the average particle size of the phenolic resin microsphere is preferably 20 to 40 μm or 30 to 50 μm.

When the dispersion medium is a hydrocarbon organic solvent (the alkane, cycloalkane), the average particle size of the phenolic resin microsphere is preferably 20 to 40 μm.

When the dispersion medium is an oil organic solvent (the silicone oil, heat transfer oil, corn oil, peanut oil, olive oil and industrial white oil), the average particle size of the phenolic resin microsphere is preferably 30 to 50 μm.

The technical solution 5 provided by the present invention is: a porous carbon microsphere. The porous carbon microsphere has a mesopore structure, the mesopore has an average pore size of 2 to 15 nm, preferably 5 to 10 nm; and the porous carbon microsphere has a specific surface area of 6 to 30 m²/g, preferably 10 to 30 m²/g. Preferably, the porous carbon microsphere has a particle size of 10 to 60 μm, further preferably 20 to 50 μm. Preferably, the method for preparing a porous carbon microsphere comprises the step of: calcining carbonization of resin microsphere (e.g. phenolic resin microsphere or polystyrene microsphere). The specific reaction conditions and parameters are described above.

The technical solution 6 provided by the present invention is: a radioactive carbon microsphere. The radioactive carbon microsphere is produced by the preparation method as previously described.

The technical solution 7 provided by the present invention is: use of the radioactive carbon microsphere as previously described in the preparation of medicament for the treatment of tumor.

The technical solution 8 provided by the present invention is: use of the radioactive carbon microsphere as previously described in the preparation of radiopharmaceutical.

The technical solution 9 provided by the present invention is: a method for treating tumor or cancer, specifically by administering a therapeutically effective amount of radioactive carbon microsphere to a subject in need thereof. The radioactive carbon microsphere is as described above.

As used herein, the term “therapeutically effective amount” means a dose that is sufficient to treat or alleviate the disease or symptom in a relatively safe manner. Typically, the therapeutically effective amount may vary with the subject's medical history, age, condition, gender, and the severity and type of the subject's medical condition, as well as the administration of other pharmacologically active agents, and those skilled in the art may determine the appropriate effective amount based on, for example, the foregoing.

On the basis of common knowledge in the art, each of the above preferred conditions can be arbitrarily combined to obtain each of the preferred examples of the present invention.

The reagents and raw materials used in the present invention are commercially available.

The positive benefit effects of the present invention are as follows.
(1) By controlling the BET multi-point specific surface area and mesopore average pore size of the prepared porous carbon microsphere, the present invention effectively enhances the loading rate of the porous carbon microsphere for radioactive precipitate and reduces the static loss rate and vibration loss rate, so as to improve the safety of the radioactive carbon microsphere during transportation and storage, and to enhance the possibilities of the radioactive carbon microsphere for medical use.
(2) The method for preparing a phenolic resin microsphere in the present invention prepares phenolic resin microsphere by stirring and mixing the reaction monomer, catalyst, and dispersion medium, and then carrying out a reaction. Compared with the prior art process in which the reactive monomer is first pre-polymerized under the action of a catalyst, and then the pre-polymer is added to a dispersion medium to form spheres, the steps and post-treatment of the preparation process of the present invention are simple, with a good control of the particle size, which is suitable for use in interventional therapy of hepatic arterial radio-embolization, and with a uniform distribution of the sphere diameter and a high yield.
(3) The method for preparing a phenolic resin microsphere of the present invention does not require the use of curing agent and pore-forming agent, etc., which reduces the use of non-essential material and lowers the difficulty of the process treatment, for example, hexamethylenetetramine is an explosive hazardous chemical, and non-use of such substance reduces the risk in the production process.
(4) Using the phenolic resin microsphere prepared in the present invention is conducive to the preparation of porous carbon material microsphere with controllable particle size, uniform distribution of sphere diameter and high strength, which is more suitable for the loading of nuclide and has a high loading rate and a low loss rate, which can ensure the safety of medication.

### BRIEF DESCRIPTION OF DRAWINGS

FIG. 1 shows the particle size distribution of the phenolic resin microspheres produced in Example I-4.

FIG. 2 shows a scanning electron micrograph (SEM) of the porous carbon material microspheres produced in Example 2-(1).

FIG. 3 shows a scanning electron micrograph (SEM) of the porous carbon material microspheres produced in Example 2-(2).

FIG. 4 shows the particle size distribution of the phenolic resin microspheres produced in Example II-2.

FIG. 5 shows the particle size distribution of the porous carbon microspheres in Example 2-(3).

FIG. 6 shows a curve graph of the combined pore volume-pore size distribution of the porous carbon microsphere BJH desorption in Example 2-(3).

FIG. 7 shows a scanning electron micrograph (SEM) of the porous carbon microspheres in Example 2-(4).

FIG. 8 shows the particle size distribution of the porous carbon microspheres in Example 2-(4).

FIG. 9 shows a curve graph of the combined pore volume-pore size distribution of the porous carbon microsphere BJH desorption in Example 2-(4).

### SPECIFIC EMBODIMENT OF THE INVENTION

The solutions of the present invention will be explained below in connection with examples. It will be understood by those skilled in the art that the following examples are intended to illustrate the invention only and should not be considered as limiting the scope of the invention. Where specific techniques or conditions are not indicated in the examples, follow the techniques or conditions described in the literature in the art or in accordance with the product specification. The reagents or instruments used without indication of the manufacturer are conventional products that are commercially available. The formaldehyde used in the following examples is 37% aqueous solution of formaldehyde.

### EXPLANATION OF TERMS

In the present invention, “porous carbon material microsphere” and “porous carbon microsphere” have the same meaning, and “radioactive carbon microsphere” and “radioactive carbon material microsphere” have the same meaning.

In the present invention, “the complex containing radionuclide is distributed within the pores of the mesopore” can be understood to mean that the complex containing radionuclide is partially or wholly distributed within the pores of the mesopore, or it can be understood to mean that the pores of the mesopore are distributed with the complex containing radionuclide.

In the present invention, the terms “precipitate”, “complex”, and “complex precipitate” have the same meaning, and all refer to a precipitate immobilized with a metallic element (e.g., a radioactive element) that is produced by reaction with a precipitating agent.

In the present invention, the term “precipitating agent” means a substance capable of reacting with a radionuclide to form particle of significantly reduced solubility, such as solid precipitate that can be formed by various types of reactions, such as complexation, chelation, association, and salting out reactions. The precipitating agent is selected from tartaric acid, EDTA, sodium phosphate, 8-hydroxyquinoline, aromatic carboxylic acid, quinolones, flavonols, N,N′-bis(2-hydroxyphenyl)oxalic acid diamide, dibutyl methylphosphonate (DPMA), trifluoromethanesulfonic acid, 2,6-di-tert-butyl-4-methylphenol, asparagine/glutamine, oxalate, thiocyanate, pyrophosphate, 5-fluorouracil, methacrylic acid/sorbic acid/salicylic acid and 8-hydroxyquinoline, aromatic carboxylic acids and 1,10-o-phenanthroline/4,4′-bipyridine, oxalic acid and o-phenanthroline, phenylalanine and o-phenanthroline, phosphates and serum proteins, nicotinic acid with 8-hydroxyquinoline, isophthalic acid and acetylacetone, indole-3-propionic acid and o-phenanthroline, aspartic acid and o-phenanthroline, Schiff base and o-phenanthroline, etc.

The applicant first explored the preparation of phenolic resin microspheres with the aim of obtaining phenolic resin microspheres with better sphericity and the required average particle size at a higher yield.

### Example 1. Preparation of Phenolic Resin Microspheres

(Example I-1 to Example I-15: Preparation of phenolic resin microspheres using a hydrocarbon organic solvent as a dispersion medium; Example II-1 to Example II-8: Preparation of phenolic resin microspheres using an oil organic solvent as a dispersion medium)

### Example I-1 to Example I-15. Preparation of Phenolic Resin Microspheres Using Hydrocarbon Organic Solvent as a Dispersion Medium

The phenolic compound was mixed with deionized water by magnetic stirring, and added with the deionized water solution of basic catalyst, formaldehyde and dispersion medium, and the mixture was obtained after magnetic stirring for 30 minutes; the mixture was transferred to an autoclave, and after reacting for 48 hours at a certain temperature, washed by anhydrous ethanol and deionized water, dried in vacuum at 60 °C, and sieved, to give phenolic resin microspheres. The specific process parameters and reaction results are shown in Table 1 below.

TABLE 1

| | | | | Molar | | | | | |
|---|---|---|---|---|---|---|---|---|---|
| | | | | ratio of | | | | | |
| | Types and | | | phenolic | | Types and | | | |
| | amounts of | | Types of | compound | | amounts of | Magnetic | | |
| | phenolic | Formaldehyde/ | basic | to basic | Deionized | dispersion | stirring | Reaction | |
| No. | compounds | mL | catalysts | catalyst | water/mL | medium | speed/rpm | temperature/°C | Results |
| Example | resorcinol | 14 | Sodium | 100:0.01 | 40 | n-Heptane | 200 | 90 | Using n-heptane as |
| I-1 | 6.4 g | | carbonate | | | 30 mL | | | the dispersant, |
| Example | resorcinol | 14 | Sodium | 100:0.01 | 40 | n-Heptane | 200 | 90 | formaldehyde, |
| I-2 | 6.4 g | | carbonate | | | 30 mL | | | basic catalyst, |
| Example | resorcinol | 14 | Sodium | 100:0.005 | 30 | n-Heptane | 200 | 85 | dispersant, and |
| I-3 | 6.4 g | | carbonate | | | 60 mL | | | parameters such as |
| Example | resorcinol | 14 | Sodium | 100:0.005 | 30 | n-Heptane | 200 | 85 | reaction |
| I-4 | 6.4 g | | carbonate | | | 90 mL | | | temperature and |
| Example | resorcinol | 14 | Sodium | 100:0.005 | 30 | n-Heptane | 200 | 85 | stirring rate were |
| I-5 | 6.4 g | | carbonate | | | 120 mL | | | explored, and the |
| Example | resorcinol | 14 | Calcium | 100:0.005 | 30 | n-Heptane | 50 | 85 | overall sphericity |
| I-6 | 6.4 g | | hydroxide | | | 120 mL | | | was good, with the |
| Example | resorcinol | 14 | Calcium | 100:0.005 | 30 | n-Heptane | 100 | 85 | particle size |
| I-7 | 6.4 g | | hydroxide | | | 120 mL | | | concentrated in 20 |
| Example | resorcinol | 14 | Calcium | 100:0.005 | 30 | n-Heptane | 150 | 85 | μm to 40 μm, and |
| I-8 | 6.4 g | | hydroxide | | | 120 mL | | | the yield could |
| Example | resorcinol | 20 | Calcium | 100:0.1 | 50 | n-Heptane | 300 | 85 | reach 70% to 80%. |
| I-9 | 6.4 g | | hydroxide | | | 120 mL | | | |
| Example | phenol | 14 | Calcium | 100:0.05 | 40 | n-Heptane | 200 | 85 | |
| I-10 | 6.4 g | | hydroxide | | | 120 mL | | | |
| Example | phenol | 9 | Sodium | 100:0.001 | 20 | n-Heptane | 200 | 85 | |
| I-11 | 6.4 g | | carbonate | | | 120 mL | | | |
| Example | phenol | 9 | Sodium | 100:0.005 | 30 | n-Heptane | 200 | 75 | |
| I-12 | 6.4 g | | carbonate | | | 60 mL | | | |
| Example | resorcinol | 9 | Sodium | 100:0.005 | 30 | n-Heptane | 200 | 60 | |
| I-13 | 6.4 g | | carbonate | | | 120 mL | | | |
| Example | resorcinol | 11 | Sodium | 100:0.005 | 30 | Cyclohexane | 200 | 60 | The overall |
| I-14 | 6.4 g | | carbonate | | | 120 mL | | | sphericity was |
| Example | resorcinol | 9 | Sodium | 100:0.001 | 30 | Cyclohexane | 200 | 70 | good, and the |
| I-15 | 6.4 g | | hydroxide | | | 90 mL | | | microsphere size |
| | | | | | | | | | was concentrated |
| | | | | | | | | | in 20 μm to 40 μm, |
| | | | | | | | | | and the yield was |
| | | | | | | | | | 72% to 76%. |

Note:
The volume of deionized water in the above table includes the total volume of deionized water in “Mixing phenolic compound with deionized water” and “Deionized water solution of basic catalyst”.

### Comparative Examples 1 to 4. Preparation of Phenolic Resin Microspheres Using Small Molecular Surfactant as a Dispersion Medium

Under other experimental conditions identical or essentially identical to those of Example I, the preparation of phenolic resin microspheres was carried out by adding a small molecular surfactant to an alkane or cycloalkane dispersion medium or by directly employing a small molecular surfactant dispersion medium, and the specific process parameters and results are shown in Table 2.

TABLE 2

| | Types and | | | Molar ratio | | | | | |
|---|---|---|---|---|---|---|---|---|---|
| | amounts | | | of phenolic | | Types and | Magnetic | | |
| | of | | Types of | compound | | amounts of | stirring | Reaction | |
| | phenolic | Formaldehyde/ | basic | to basic | Deionized | dispersion | speed/ | temperature/ | |
| No. | compounds | mL | catalysts | catalyst | water/mL | medium | rpm | °C | Results |
| Comparative | resorcinol | 14 | Sodium | 100:0.005 | 30 | n-heptane | 200 | 85 | The size of |
| example 1 | 6.4 g | | carbonate | | | 60 mL + | | | phenolic resin |
| | | | | | | 0.1 g Span − | | | microspheres |
| | | | | | | 80 | | | had a greater |
| | | | | | | | | | degree of |
| | | | | | | | | | reduction and |
| | | | | | | | | | cannot reach |
| | | | | | | | | | the micrometer |
| | | | | | | | | | scale. |
| Comparative | resorcinol | 14 | Sodium | 100:0.005 | 30 | without | 200 | 85 | No dispersion |
| example 2 | 6.4 g | | carbonate | | | adding | | | medium added, |
| | | | | | | | | | not forming a |
| | | | | | | | | | spherical shape. |
| Comparative | resorcinol | 14 | Sodium | 100:0.005 | 30 | Polyvinyl | 300 | 85 | The overall |
| example 3 | 6.4 g | | carbonate | | | alcohol 2 g | | | reaction was |
| | | | | | | | | | heavily |
| | | | | | | | | | agglomerated |
| | | | | | | | | | and did not |
| | | | | | | | | | form a spherical |
| | | | | | | | | | shape. |
| Comparative | resorcinol | 9 | Sodium | 100:0.005 | 30 | n-heptane | 200 | 85 | The overall |
| example 4 | 6.4 g | | carbonate | | | 120 mL + | | | reaction was |
| | | | | | | polyvinyl | | | heavily |
| | | | | | | alcohol | | | agglomerated |
| | | | | | | 1.0 g | | | and did not |
| | | | | | | | | | form a spherical |
| | | | | | | | | | shape. |

Note:
The volume of deionized water in the above table includes the total volume of deionized water in “Mixing phenolic compound with deionized water” and “Deionized water solution of basic catalyst”.

Through investigations of the aforementioned method for preparing phenolic resin, it was found that the choice of dispersion medium profoundly affects both the microsphere formation efficiency and particle size of phenolic resin, the dispersion medium are selected from alkane, and cycloalkane; and the exclusion of small-molecular surfactant in the dispersion medium enables the production of phenolic resin microspheres with average particle size ranging from 20 m to 40 μm in higher yield. The addition of small molecular surfactant resulted in a greater reduction in microsphere size and failure to reach the target particle size range. Without the addition of dispersant, it is difficult to prepare the product into a spherical shape.

On this basis, the inventors also tried to improve the viscosity of the dispersion medium to improve the particle size of the microspheres, such as the use of high-viscosity polyvinyl alcohol to form a dispersion system, but the result was that the overall size of the microspheres was not significantly improved, and resulted in a large number of agglomerations in the reaction system, and the yield of phenolic resin microspheres was extremely low.

### Examples II-1 to II-8. Preparation of Phenolic Resin Microspheres Using an Oil Organic Solvent as a Dispersion Medium

The phenolic compound was mixed with deionized water by magnetic stirring, and added with the deionized water solution of basic catalyst, formaldehyde and dispersion medium, and the mixture was obtained after magnetic stirring at 70 to 400 rpm/min for 30 minutes; the mixture was transferred to an autoclave, and after reacting at 60 to 100 °C for 48 hours, washed by anhydrous ethanol and deionized water, dried in vacuum at 60 °C, and sieved, to give phenolic resin microspheres. Specific reaction parameters are shown in Table 3.

TABLE 3

| | | | | Molar ratio | | | | | |
|---|---|---|---|---|---|---|---|---|---|
| | Types and | | | of phenolic | | Types and | | | |
| | amounts of | | Types of | compound | | amounts of | Magnetic | Reaction | |
| | phenolic | Formaldehyde/ | basic | to basic | Deionized | dispersion | stirring | temperature/ | |
| No. | compounds | mL | catalysts | catalyst | water/mL | medium | speed/rpm | °C | Results |
| Example | resorcinol | 9 | Sodium | 100:0.01 | 40 | Silicone oil | 200 | 85 | The overall |
| II-1 | 6.4 g | | carbonate | | | 20 mL | | | sphericity |
| Example | resorcinol | 9 | Sodium | 100:0.005 | 25 | Silicone oil | 200 | 85 | was good, the |
| II-2 | 6.4 g | | carbonate | | | 40 mL | | | microsphere |
| Example | resorcinol | 9 | Sodium | 100:0.001 | 20 | Silicone oil | 200 | 60 | particle size |
| II-3 | 6.4 g | | carbonate | | | 40 mL | | | was |
| Example | phenol | 9 | Sodium | 100:0.01 | 30 | Silicone oil | 200 | 100 | concentrated |
| II-4 | 6.4 g | | carbonate | | | 40 mL | | | in 30 μm to |
| Example | phenol | 14 | Sodium | 100:0.005 | 30 | Silicone oil | 200 | 85 | 50 μm, the |
| II-5 | 6.4 g | | carbonate | | | 40 mL | | | yield was |
| Example | phenol | 9 | Sodium | 100:0.005 | 30 | Silicone oil | 200 | 85 | 65% to 85% |
| II-6 | 6.4 g | | carbonate | | | 40 mL | | | on average, |
| Example | resorcinol | 9 | Sodium | 100:0.005 | 30 | Industrial | 200 | 85 | and can reach |
| II-7 | 6.4 g | | carbonate | | | white oil | | | more than |
| Example | resorcinol | 9 | Sodium | 100:0.005 | 30 | Olive Oil | 200 | 85 | 80% by use |
| II-8 | 6.4 g | | carbonate | | | 40 mL | | | of silicone oil |
| | | | | | | | | | as a |
| | | | | | | | | | dispersant. |

Note:
The volume of deionized water in the above table includes the total volume of deionized water in “Mixing phenolic compound with deionized water” and “Deionized water solution of basic catalyst”.

### Comparative Example 5. Preparation of Phenolic Resin Microspheres by Adding Surfactants to a Dispersion Medium System of an Oil Organic Solvent

Phenolic resin microspheres were prepared under other experimental conditions that were the same or essentially the same as those in Example II, and the specific reaction parameters are shown in Table 4.

TABLE 4

| | | | | Molar ratio | | | | | |
|---|---|---|---|---|---|---|---|---|---|
| | Types and | | | of phenolic | | Types and | | | |
| | amounts of | | Types of | compound | | amounts of | Magnetic | Reaction | |
| | phenolic | Formaldehyde/ | basic | to basic | Deionized | dispersion | stirring | temperature/ | |
| No. | compounds | mL | catalysts | catalyst | water/mL | medium | speed/rpm | °C | Results |
| Comparative | resorcinol | 9 | Sodium | 100:0.005 | 30 | Silicone oil | 85 | 150 | The product |
| example 5 | 6.4 g | | carbonate | | | 40 mL + CTAB | | | was in the |
| | | | | | | 0.1 g | | | form of |
| | | | | | | | | | wafers, not |
| | | | | | | | | | spherical. |

Note:
The volume of deionized water in the above table includes the total volume of deionized water in “Mixing phenolic compound with deionized water” and “Deionized water solution of basic catalyst”.

The inventors explored the use of only oil as a dispersion system, and found that the sphericity was better, the microsphere particle size had a slight increase, and the yield of 30 to 50 μm microspheres could reach 65% to 85%. The product prepared by adding the small molecular surfactant CTAB to the silicone oil system is wafer-like and not spherical.

### Example 2. Preparation of Porous Carbon Microspheres

The applicant further explored the preparation of porous carbon microspheres using phenolic resin microspheres as raw material after obtaining an ideal phenolic resin through the exploration of the above experiments.

### Example 2-(1). Preparation of Porous Carbon Material Microspheres

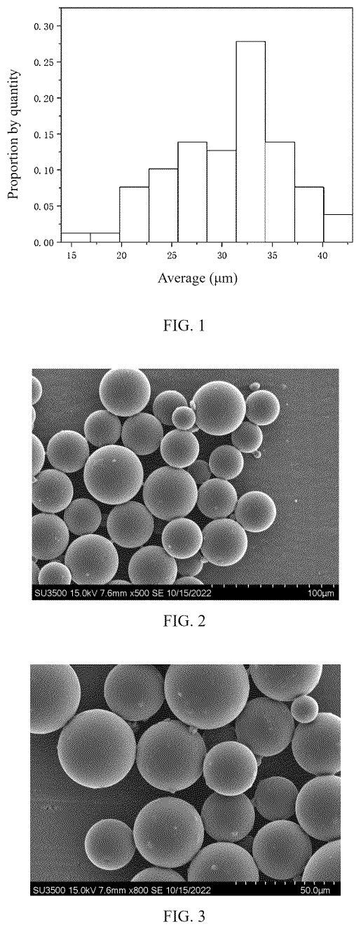

The porous carbon material microspheres were obtained by calcining the phenolic resin microspheres prepared in Example I-4 (the particle size distribution diagram is shown in FIG. 1) under Ar atmosphere at 700 °C for 4 h (the heating rate was 3 °C/min); the porous carbon material microspheres were washed with 0.3 mol/L of dilute nitric acid, and deionized water for 2 times in sequence, dried, sieved, to obtain the porous carbon material microspheres (the scanning electron micrograph is shown in FIG. 2); the BET multi-point specific surface area of the porous carbon material microspheres in Example 2-(1) was 10.01 m²/g, and the average pore size of mesopores was 9.32 nm.

### Example 2-(2). Preparation of Porous Carbon Material Microspheres

The porous carbon material microspheres were obtained by calcining the phenolic resin microspheres prepared in Example I-15 under Ar atmosphere at 800 °C for 4 h (the heating rate was 3 °C/min); the porous carbon material microspheres were washed with 0.3 mol/L of dilute nitric acid, and deionized water for 2 times in sequence, dried, sieved, to obtain the porous carbon material microspheres (the scanning electron micrograph is shown in FIG. 3); the BET multi-point specific surface area of the porous carbon material microspheres in Example 2-(2) was 22.32 m²/g, and the average pore size of mesopores was 6.75 nm.

### Example 2-(3). Preparation of Porous Carbon Material Microspheres

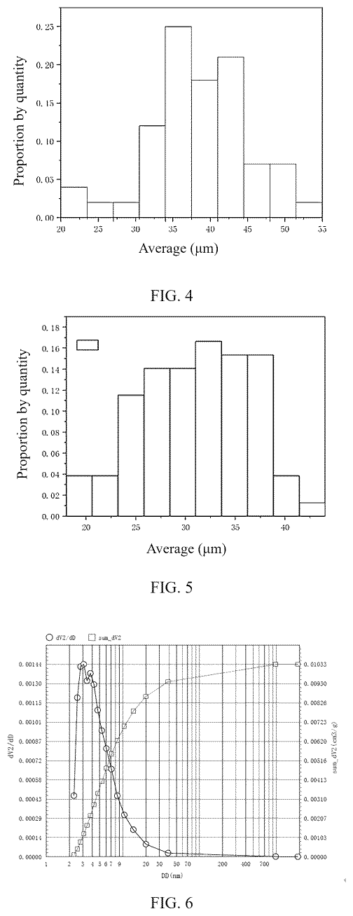

The porous carbon microspheres were obtained by calcining the phenolic resin microspheres prepared in Example II-2 (the particle size distribution diagram is shown in FIG. 4) under Ar atmosphere at 800 °C for 4 h; wherein the heating rate was 3 °C/min during the process of elevating temperature from room temperature to 800 °C.

The particle size distribution of the porous carbon microspheres prepared in Example 2-(3) is shown in FIG. 5, and the average particle size distribution was concentrated in 25 to 38 μm.

The porous carbon microspheres produced in Example 2-(3) had a BET multi-point specific surface area of 14.5 m²/g, a distinct mesoporous structure, a relatively uniform mesoporous pore size distribution, and an average mesoporous pore size of 6.44 nm, as identified by the Precision Micro Gobo JW-BK112 mesopore analyzer. A curve graph of the combined pore volume-pore size distribution of the porous carbon microsphere BJH desorption in Example 2-(3) is shown in FIG. 6.

### Example 2-(4). Preparation of Porous Carbon Material Microspheres

Preparation of porous carbon microspheres: Polystyrene microspheres (purchased from DuPont AmberChrom™ XT30, USA) were calcined under Ar atmosphere at 400 °C for 4 h (the temperature was elevated from room temperature to 200 °C at 10 °C/min, then to 400 °C at 3 °C/min, kept for 240 min, and then cooled down at the rate of 10 °C/min), to prepare the porous carbon microspheres.

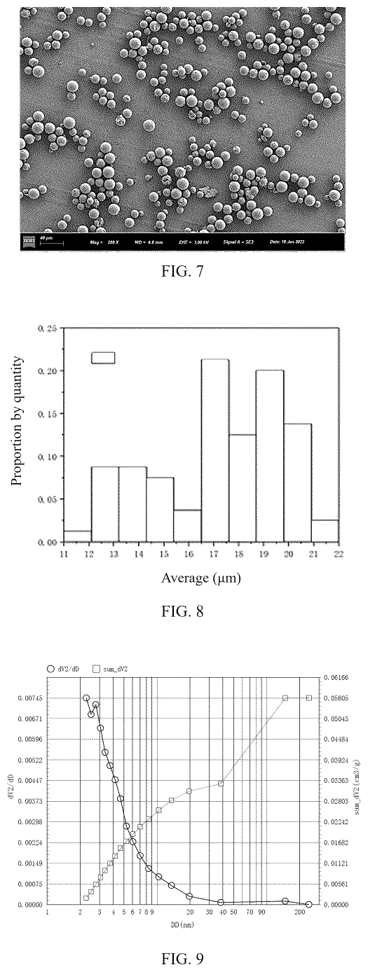

SEM image of the porous carbon microspheres prepared in Example 2-(4) is shown in FIG. 7; the average particle size was concentrated at 17 to 21 μm, as shown in FIG. 8.

The porous carbon microspheres produced in Example 2-(4) had a BET multi-point specific surface area of 25.5 m²/g, a distinct mesoporous structure, a relatively uniform mesoporous pore size distribution, and an average mesoporous pore size of 8.48 nm, as identified by the Precision Micro Gobo JW-BK112 mesopore analyzer. A curve graph of the combined pore volume-pore size distribution of the porous carbon microsphere BJH desorption in Example 2-(4) is shown in FIG. 9.

### Example 3. Preparation of Phenolic Resin Microspheres and Porous Carbon Material Microspheres (Scale-Up Production)

640 g of resorcinol was mixed well with 3 L of deionized water, added with sodium carbonate aqueous solution (62 g of sodium carbonate/0.5 L of deionized water), 1.1 L of formaldehyde and 10 L of n-heptane, and magnetically stirred at 200 rpm/min for 30 minutes to obtain a mixture; the mixture was transferred to the autoclave for reacting at 60 °C for 72 hours. After completion of the reaction, the phenolic resin microspheres were obtained through solid-liquid separation, cleaned by n-heptane and ethanol, dried by rotary evaporator, and dried by blast drying oven to obtain phenolic resin microspheres (the particle sizes were concentrated in 20 μm to 40 μm).

The prepared phenolic resin microspheres were placed in a tube furnace, protected by argon atmosphere, and carbonized at 800 °C for 4 h to obtain porous carbon material microspheres, which were sequentially cleaned with dilute nitric acid and purified water, placed in a blast drying oven for drying, and sieved by a sifter, to obtain porous carbon material microspheres; the particle sizes were concentrated in 20 μm to 40 μm, and the yield was 76.2%.

### Comparative Example 6. Preparation of Phenolic Resin Microspheres and Porous Carbon Material Microspheres

Carbon microspheres were prepared by the preparation method disclosed in Feng Chong, Zhao Jianghong, Han Baixin et al. Emulsifier-free preparation of carbon microspheres by reversed-phase emulsion method and their electrochemical properties[J]. New Carbon Materials, 2016, 31(6): 600-608, and the specific method is as follows:

Heat transfer oil and silicone oil were mixed at a mass ratio of 4:1, and preheated and stirred at 115 °C for 1 h. A certain amount of thermosetting phenolic resin and ethanol (mass ratio 1:4) were weighted, mixed and stirred well, the mixture solution was slowly poured into the preheated mixing oil, stirred at 115 °C for 2 h (stirring rate of 500 r/min), followed by filtration, separation, washing, drying, to obtain phenolic resin microspheres.

The obtained phenolic resin microspheres were carbonized at 800 °C under Ar atmosphere for 1 h to obtain porous carbon microspheres. The average particle size of the prepared porous carbon spheres was concentrated at 10 to 20 μm, the BET multi-point specific surface area was 540 m²/g, and the average pore size of mesopores was 0.89 nm.

### Comparative Example 7. Preparation of Phenolic Resin Microspheres and Porous Carbon Material Microspheres

The mesitylene (1 eq), resorcinol (1 eq) and formaldehyde (1 eq) were added to the deionized water and stirred to dissolve, and the mixture was first stirred at high speed of 800 r/min for 5 min, and then at low speed of 100 r/min for 5 min to prepare the precursor solution. The precursor solution was added to the industrial white oil containing a volume fraction of 1% of the span-80 (the volume ratio of the precursor solution to industrial white oil was 1:8), stirred and mixed well, then reacted at 25 °C in an oil bath for 12 h to produce a turbid solution; the turbid solution was suction filtered and separated, and washed with dichloromethane for several times to obtain a powdery solid. The powdered solid was carbonized at 850 °C for 5 h to obtain porous carbon microspheres. The particle size of the prepared carbon spheres was concentrated from 1 to 10 μm, with a BET multi-point specific surface area of 930 m²/g and a mesopore average pore size of 18.56 nm.

By using phenolic resin microspheres with an average particle size in the range of 20 to 50 μm, the prepared carbon microspheres have a specific surface area of 6 to 30 m²/g, preferably 10 to 30 m²/g, and the carbon microspheres with the average particle size controlled to be 1 to 150 μm, preferably 10 to 60 μm, with an average pore size in the range of 2 to 15 nm. In order to enhance the loading rate of radioactive precipitates on porous carbon microspheres and reduce their static loss rate and vibration loss rate, this is realized by controlling the BET multi-point specific surface area and average pore size of mesoporous of the porous carbon microspheres.

As above, the applicant prepared and obtained porous carbon material microspheres with an average particle size of 10 to 60 μm, a specific surface area of 10 to 30 m²/g, and a mesoporous pore size of 5 to 10 nm, in order to load radionuclides with a high loading rate and a low static loss rate and vibration loss rate, the applicant validated the effect of loading nuclides of the prepared and obtained carbon microspheres as described above.

### Effect Example

The porous carbon microspheres prepared in the above-described Example 2, comparative examples 6 and 7 were immersed in 0.3 mol/L of dilute nitric acid for 2 h and subsequently washed with deionized water for three times in sequence, dried, and then used for loading complex containing radionuclides.

0.15 g of porous carbon microspheres were mixed with precipitating agent solution and YCl₃ solution to obtain the reaction solution, with a volume of 3 mL, and could be reacted and loaded with nuclides according to any of the following modes (the types of precipitating agents are shown in Tables 5 to 7):
Mode 1: Adding YCl₃ solution and porous carbon microspheres first, so that YCl₃ fully entering into the mesopores of the porous carbon microspheres, and then adding the precipitating agent solution, which reacting in the mesopores of the porous carbon microspheres to generate complex; and
Mode 2: Adding the precipitating agent solution and porous carbon microspheres first, so that the precipitating agent fully entering the mesopores of the porous carbon microspheres, and then adding YCl₃ solution, which reacting in the mesopores of the porous carbon microspheres to generate complex. The details are as follows:

The porous carbon microspheres were added to a solution containing a precipitating agent to obtain a mixture solution, mixed well and left to stand for 12 h. Then, YCl₃ solution was added to the mixture solution and shaken in a shaker for 1 h, which allowed the complex containing radionuclides to be fully loaded onto the porous carbon microspheres.

When the precipitating agents were tartaric acid and sodium phosphate, the Y-tartaric acid complex-loaded porous carbon microspheres were first added to the sodium phosphate solution, shaken in a shaker for 1 h, washed and dried to produce Y—PO₄ complex-loaded porous carbon microspheres (EDTA was EDTA-2Na).

The reacted porous carbon microsphere solution was suction filtered through deionized water three times to determine the final loading rate.

Half of the collected porous carbon microspheres were immersed in saline for 6 days. The yttrium ion content of the solution was determined to calculate the static loss rate.

The other half of the collected porous carbon microspheres were immersed in saline for 6 days and shaken in a shaker for 30 min every 12 h. The yttrium ion content of the solution was determined to determine the vibration loss rate.

TABLE 5

| | | | | Loading | Static loss | Vibration |
|---|---|---|---|---|---|---|
| | Porous carbon | Tartaric acid | YCl₃ concentration | rate | rate | loss rate |
| No. | microspheres | concentration g/mL | g/mL | % | % | % |
| 1 | Example 2-(1) | 0.025 | 0.0125 | 94.5 | <0.1 | 1.4 |
| 2 | Example 2-(2) | 0.025 | 0.0125 | 96.3 | <0.1 | 1.3 |
| 3 | Example 2-(3) | 0.025 | 0.0125 | 93.5 | <0.1 | 2.1 |
| 4 | Example 2-(3) | 0.0167 | 0.0083 | 96.2 | <0.1 | 1.5 |
| 5 | Comparative example 6 | 0.025 | 0.0125 | 85.8 | <0.1 | 1.3 |
| 6 | Comparative example 7 | 0.025 | 0.0125 | 92.6 | 0.3 | 8.6 |

TABLE 6

| | | | Sodium | | | | |
|---|---|---|---|---|---|---|---|
| | | Tartaric acid | phosphate | YCl₃ | Loading | Static | Vibrational |
| | Porous carbon | concentration | concentration | concentration | rate | loss rate | loss rate |
| No. | microspheres | g/mL | g/mL | g/mL | % | % | % |
| 1 | Example 2-(4) | 0.025 | 0.0115 | 0.0125 | 91.4 | <0.1 | 1.4 |
| 2 | Example 2-(4) | 0.0167 | 0.0459 | 0.0083 | 90.4 | <0.1 | 1.2 |
| 3 | Example 2-(4) | 0.0167 | 0.0077 | 0.0083 | 96.1 | <0.1 | 0.8 |
| 4 | Comparative | 0.0167 | 0.0459 | 0.0083 | 83.5 | <0.1 | 0.9 |
| | example 6 | | | | | | |
| 5 | Comparative | 0.0167 | 0.0459 | 0.0083 | 93.2 | 0.4 | 7.2 |
| | example 7 | | | | | | |

TABLE 7

| | | EDTA | YCl₃ | | Static loss | Vibrational |
|---|---|---|---|---|---|---|
| | Porous carbon | concentration | concentration | Loading rate | rate | loss rate |
| No. | microspheres | g/mL | g/mL | % | % | % |
| 1 | Example 2-(3) | 0.103 | 0.023 | 90.4 | <0.1 | 0.5 |
| 2 | Example 2-(3) | 0.051 | 0.0114 | 91.6 | <0.1 | 1.1 |
| 3 | Example 2-(3) | 0.034 | 0.0076 | 93.1 | <0.1 | 1.2 |
| 4 | Comparative example 6 | 0.034 | 0.0076 | 84.7 | <0.1 | 1.7 |
| 5 | Comparative example 7 | 0.034 | 0.0076 | 94.8 | 0.3 | 9.1 |

It can be seen through Tables 5 to 7 that:
(1) Considering that the half-life of ⁹⁰Y is 64.2 h, and that the ⁹⁰Y products usually used in medicine are generally only allowed to be used at intervals of no more than 5-6 days from the production of the product to the clinical application in actual application, the indicators of static loss rate and vibration loss rate with a period of 6 days were set up in order to examine the degree of quality and safety control during the regular storage period and the extreme storage period. In addition, the level of loading rate also affects the production cost and post-treatment process complexity to a certain extent, so it is examined together.

The inventors were surprised to find that the loading rate and loss rate of the porous carbon microspheres to the complex precipitating agent were affected by the mesopore size. The mesopore sizes of the porous carbon microspheres in comparative examples 6 and 7 fall outside the protection scope of the present invention, as can be seen in Tables 5 to 7, the loading rate of comparative example 6 is generally low, whereas the static loss rate and vibration loss rate of comparative example 6 are generally high. Without wishing to be bound by any specific theory, based on the present experimental results, the inventors believe that the mesopore size of the porous carbon microspheres affects the loading and the degree of loading firmness. When the mesopore size of porous carbon microspheres is small (e.g., less than 1 nm), their mesopore space is relatively limited, and there are certain restrictions on the reactants for entry and exit of the mesopore, as well as their ability to adsorb complexes generated outside the mesopore to precipitate is insufficient, resulting in a low overall loading rate. When the mesopore size of porous carbon microspheres is large (e.g., more than 15 nm), their mesopore space is large, and the amount of complex precipitates generated outside the mesopore that can be adsorbed into the mesopore is more sufficient, with a better loading rate, but the mosaic fixation of complex precipitates in the mesopore is relatively limited, and its loss rate rises significantly under a longer period of static time, especially vibration, which is not able to satisfy the requirements of safety. The inventors noticed for the first time the influence of the mesopore size of porous carbon microspheres on the loading of radioactive elements in this particular application scenario, and by selecting carbon microspheres with a specific range of mesopore sizes, the loading rate during manufacturing and the control level of loss rate during transportation and storage of the product are effectively improved.
(2) The inventors of the present invention have found that porous carbon material microspheres for loading metal elements should meet certain particle size requirements and mesopore sizes. However, if we want to obtain porous carbon material microspheres that are compatible with the loading of drugs or nuclides, the particle size of the organic material microspheres used as precursors has certain requirements. Using the alkanes, cycloalkanes and oils shown in Example I and Example II as dispersants, and without adding small molecular surfactant, phenolic resin microspheres can be prepared in high yields to meet the requirements of the particle size, and the sphericity is good; furthermore, by using the phenolic resin microspheres as a precursor, the obtained porous carbon microspheres have almost no change in the particle size, and the loading of metal elements maintains a better morphology and has a high strength.

1. A radioactive carbon microsphere, characterized in that the radioactive carbon microsphere comprises a porous carbon microsphere and a complex containing radionuclide; wherein the porous carbon microsphere has a mesopore structure, the mesopore has an average pore size of 2 to 15 nm, and the complex containing radionuclide is distributed within the pores of the mesopore; and the porous carbon microsphere has a specific surface area of 6 to 30 m²/g.

2. The radioactive carbon microsphere according to claim 1, characterized in that the porous carbon microsphere has a particle size of 10 to 60 μm.

3. The radioactive carbon microsphere according to claim 1, characterized in that the porous carbon microsphere has a specific surface area of 10 to 30 m²/g, and the mesopore has an average pore size of 5 to 10 nm.

4. The radioactive carbon microsphere according to claim 1, characterized in that in the complex containing radionuclide, the radionuclide is selected from at least one of ⁹⁰Y, ³²P, ¹⁹²Ir, ¹⁰³Pd, ⁸⁹Sr, ²²⁶Ra, ¹³¹I, ¹²⁵I, ¹⁸⁸Re, ¹⁸⁶Re, ¹⁵³Sm, ¹⁶⁶Ho, ¹¹¹In, ⁹⁹mTc, ¹⁹²Ir, ²²⁶Ra, ¹⁷⁷Lu, ²²⁵Ac, ²¹²Bi, ²¹³Bi and ²²³Ra.

5. The radioactive carbon microsphere according to claim 1, characterized in that the dose ratio of the porous carbon microsphere to the radionuclide is 1:(0.033 to 0.233).

6. The radioactive carbon microsphere according to claim 1, characterized in that the complex containing radionuclide is obtained by reacting a solution comprising the radionuclide with a solution comprising a precipitating agent.

7. The radioactive carbon microsphere according to claim 6, characterized in that the precipitating agent is at least one of tartaric acid, EDTA and sodium phosphate.

8. The radioactive carbon microsphere according to claim 1, characterized in that the radioactive carbon microsphere is prepared by a method satisfying at least one of the following conditions (1) to (4):
(1) a concentration of the radionuclide in the reaction solution is 0.02 to 0.15 mol/L;
(2) the solution comprising a precipitating agent is one or more of a tartaric acid solution, an EDTA solution and a sodium phosphate solution;
(3) the duration of the reaction is 0.5 to 1.5 h; and
(4) after completion of the reaction, the method further comprises the step of cleaning.

9. The radioactive carbon microsphere according to claim 1, characterized in that the porous carbon microsphere is prepared by a method comprising the steps of: calcination and carbonization of phenolic resin microsphere or polystyrene microsphere.

10. The radioactive carbon microsphere according to claim 9, characterized in that,
the atmosphere for the carbonization is an inert atmosphere;
the temperature of the carbonization is 400 to 1000 °C, preferably the temperature of the carbonization is 400 to 800 °C; and
the duration of the carbonization is 1 to 10 h, preferably the duration of the carbonization is 2 to 4 h.

11. A method for preparing a phenolic resin microsphere, characterized in that the method for preparing a phenolic resin microsphere comprises: mixing and reacting a phenolic compound, water, an aldehyde compound, a basic catalyst, and a dispersion medium to obtain the phenolic resin microsphere;
wherein the dispersion medium is selected from one or more of alkane, cycloalkane, silicone oil, heat transfer oil, corn oil, peanut oil, olive oil, and industrial white oil; and the dispersion medium is free of small molecular surfactant.

12. The method for preparing a phenolic resin microsphere according to claim 11, characterized in that the method for preparing a phenolic resin microsphere satisfies at least one of the following conditions a to n:
a. the alkane is one or more of n-pentane, n-hexane, n-heptane, and n-octane;
b. the cycloalkane is cyclohexane;
c. the mixing is mixing the phenolic compound with deionized water before adding a deionized water solution of the basic catalyst, formaldehyde, and the dispersion medium;
d. the type of the basic catalyst is an inorganic base; the inorganic base of the basic catalyst is one or more of aqueous ammonia, metal hydroxide, carbonate, and bicarbonate;
e. the basic catalyst is provided in the form of an aqueous solution thereof;
f. the molar ratio of the phenolic compound to the basic catalyst is 100:(0.001 to 0.1);
g. the phenolic compound is one or more of resorcinol, phenol, cresol, nonylphenol, arylalkylphenol, cashew nut phenol, octylphenol, bisphenol A and dimethylphenol;
h. the aldehyde compound is selected from one or more of formaldehyde, acetaldehyde, and furfural;
i. the aldehyde compound is provided in the form of an aqueous solution thereof;
j. the molar ratio of the phenolic compound to the aldehyde compound is 1:(1 to 5);
k. the weight to volume ratio of the phenolic compound to the water is 1:(3 to 8) g/mL;
l. the volume ratio of the water to the dispersion medium is 1:(0.5 to 6);
m. the phenolic resin microsphere is prepared without addition of a curing agent and/or a pore-forming agent; and
n. the mixing is mixing well under a stirring condition.

13. The method for preparing a phenolic resin microsphere according to claim 12, characterized in that the method for preparing a phenolic resin microsphere satisfies at least one of the following conditions I to VIII:
I. the metal hydroxide is NaOH, KOH or Ca(OH)₂;
the carbonate is Na₂CO₃, K₂CO₃ or Cs₂CO₃;
the bicarbonate is NaHCO₃ or KHCO₃;
the basic catalyst is one or more of NaOH, KOH, Ca(OH)₂, Na₂CO₃ and K₂CO₃;
II. the molar ratio of the phenolic compound to the basic catalyst is 100:(0.005 to 0.05);
III. the molar ratio of the phenolic compound to the aldehyde compound is 1:(2 to 4);
IV. the weight to volume ratio of the phenolic compound to the water is 1:(4.7 to 7) g/mL, preferably the weight to volume ratio of the phenolic compound to the water is 1:(5 to 6.25) g/mL;
V. the volume ratio of the water to the dispersion medium is 1:(1 to 5), or preferably the volume ratio of the water to the dispersion medium is 1:(2 to 4);
VI. the rate of the stirring is 50 to 400 rpm/min;
VII. the temperature of the reaction is 50 to 100 °C; and
VIII. the duration of the reaction is 24 to 72 h.

14. Use of the radioactive carbon microsphere according to claim 1, in the preparation of a drug for the treatment of tumor or a radiopharmaceutical.

15. A porous carbon microsphere characterized in that the porous carbon microsphere has a mesopore structure, the mesopore has an average pore size of 2 to 15 nm and a specific surface area of 6 to 30 m²/g, and the porous carbon microsphere has a particle size of 10 to 60 μm.
